(19) United States
(12) Patent Application Publication
(10) Pub. No.: US 2006/0189865 A1
(43) Pub. Date: Aug. 24, 2006

(54) Method and processor for visualization of 3D vectorial quantities with color-coded direction information

(75) Inventors: Jens Guhring, Langensendelbach (DE); Axel Schreiber, Erlangen (DE)

Correspondence Address: SCHIFF HARDIN, LLP;PATENT DEPARTMENT, 6600 SEARS TOWER, CHICAGO, IL, 60606-6473, US

(73) Assignee: Siemens Aktiengesellschaft

(21) Appl. No.: 11/355,338

(22) Filed: Feb. 15, 2006

(30) Foreign Application Priority Data
Feb. 18, 2005 (DE) 10 2005 007 571.1

(51) Int. Cl. G06K 9/00; A61B 5/05

(52) U.S. Cl. 600/410; 382/128

(57) ABSTRACT

In a method for visualization of three-dimensional vectorial quantities at a data processing device with color-coded direction information, the vectorial quantities are displayed; dependent on their direction, in different colors in a three-dimensionally or two-dimensionally projected representation on an image output unit, and at least one reference element indicating the orientation of the representation and at least one explanatory specification regarding the employed color coding are shown on the image output unit. The at least one reference element specifies the orientation of the representation is designed in color as a three-dimensional element; such that the color coding used is shown clarified by the color design of the reference element.

### BACKGROUND OF THE INVENTION

1. Field of the Invention

The present invention concerns a method for visualization of three-dimensional, vectorial quantities present and/or received at a data processing device with color-coded direction information, of the type wherein the vectorial quantities are displayed in different colors dependent on their direction in a three-dimensionally or two-dimensionally projected representation on an image output unit, with at least one reference element specifying the orientation of the representation and at least one explanatory specification regarding the color coding used being shown on the image output unit.

2. Description of the Prior Art

In the visualization of three-dimensional data, for example in medicine, geology or material testing, it is necessary to document the spatial orientation of the displayed information. For this, reference elements are used that allow conclusions about the orientation with regard to the position of the image plane or a rendered volume representation. In addition, an additional explanatory legend for the representation is frequently necessary that explains an employed color coding of three-dimensional, vectorial quantities that are shown as a vector field. This 3D legend as an explanatory specification for employed color coding is additionally shown on the image output unit with regard to the actual image representation and additionally with regard to the reference element.

This representation of the orientation via a reference element and the specification regarding the employed color coding as a 3D legend, entails the problem that occlusions can quickly arise on the image output unit , such that, under the circumstances, important information can be lost to the user operating with the image representation. The representation additionally quickly becomes unclear (complex). The implementation of the orientation specification as well as of the 3D legend simultaneously requires the use of a not-insignificant quantity of source code for the program means used for visualization. Errors thus can quickly arise.

### SUMMARY OF THE INVENTION

An object of the present invention is to provide a method for visualization that is improved in this regard.

This object is achieved in accordance with the invention by a method of the type described above wherein at least one reference element specifying the orientation of the representation is designed in color as a three-dimensional element, such that the employed color coding is shown in an explanatory manner through the color design of the reference element.

This apparatus solves the problem that conventional, independent elements, namely the reference element for specification of the orientation as well as the 3D legend for explanation of the color coding, are combined into one element. The reference elements designed as graphical elements or text markings are designed in color such that they simultaneously represent a documentation of the employed color coding. A compact representation that is simultaneously intuitive thus results. The user working at the image output unit (for example a screen or with a printout) with the shown vectorial quantities can furthermore use his or her knowledge with regard to the specification of orientations or color codings in an unlimited manner, to expand the interpretation possibilities of an original reference element used for representation of the orientation. A simpler implementation is possible by the integration of the color coding in the reference element specifying the orientation, with a reduction of the number of errors that occur due to the program that forms the basis of the image representation of the quantity.

The vectorial quantities can be values from measurements or simulations that are acquired in geology or in materials testing, for example vectors that describe the force propagation given mechanical stress, or vectors that reproduce the permeability of rock layers for fluids. A further area of application is in the field of medicine, for example in the representation of diffusion processes. The blood flow or the movement direction of muscle tissue can likewise be represented with color coding. In this case, both the anatomical orientation and the color coding are to be displayed on the image output unit that is used for image representation of the quantities, for which anatomical orientation and the color coding only a single element is necessary according to the invention.

According to the invention, the predominant (preferred) direction of a diffusion process can be shown as a vectorial quantity, in particular in diffusion tensor imaging (DTI) by means of a magnetic resonance system. In this modality, molecules move driven by diffusion, with the free path lengths available in different directions generally being different, so the movement is anisotropic. Tissue in which a directed diffusion exists is shown in color, such that the anatomical predominant direction of the diffusion is clear from the color. Given the inventive visualization of such diffusion quantities, the employed color coding is specified by the color presentation of the reference element that simultaneously reproduces the anatomical orientation of the exposure. A visualization of diffusion quantities can ensue analogously in the technical field.

Cubes and/or spheres and/or cuboids and/or ellipsoids can be used as reference elements. Elements with curved surfaces (such as the sphere or an ellipsoid) on which color transitions can be indicated particularly are suitable as reference elements that can simultaneously serve for representation of a complicated color coding. Reference elements with even surfaces have the advantage that each flat surface can be associated with a specific orientation specification, for example the specification for right and left or forward and rear in the anatomy.

Given the use of a cuboid with different edge lengths or an ellipsoid, the resolution or a specific distortion of the representation of the vectorial quantities on the image output unit (such as a screen or a paper printout) can be reproduced; and a more precise representation of the color coding can ensue, if needed, in a specific direction.

According to the invention, edges and/or surfaces of the reference element and/or segmentation surfaces (in particular arising by the introduction of degrees of latitude and longitude or triangulation) of the reference element are designed in color. For example, the edges of a cube can be colored corresponding to the colors that are associated with a horizontal or vertical direction. The surfaces of a cube (or the segmentation surfaces from which a sphere is designed) can likewise be patterned in color corresponding to the color coding.

Furthermore, the reference element can embody appropriately oriented text components specifying the representation, the text components being designed in color for explanation of the color coding. For example, given an anatomical representation it is thus possible to pattern the designation “A” (which stands for “anterior”) in color corresponding to the color associated with this direction. Text components that are used anyway in order to specify an orientation (for example “vertical” or “horizontal”) can thus be designed in color, such that they can additionally be used for communication of the information with regard to the employed color coding.

Primary colors used for coding of primary directions in the representation can be represented on the respective reference element corresponding to the respective direction. For example, in medicine the primary direction anterior-posterior is frequently coded with the color green, left-right can be shown with red, head-foot with blue. These or other primary colors used for coding of primary directions are represented on the reference element corresponding to the respective direction, such that, for example, the surface of a cube that is labeled with “A” for “anterior” for specification of the orientation in an anatomical mapping is colored green. Horizontally-situated edges of a cuboid corresponding to the direction from left to right can analogously be colored red. The north pole of a spherical element is accordingly to be colored blue in order to express alignment parallel or anti-parallel to the head-foot direction.

The primary directions can form an orthogonal trihedron. This corresponds to convention in medical applications and applications in the natural sciences and engineering sciences, in which for the most part an orthogonal xyz-coordinate system is used for the primary directions.

In an embodiment of the invention combination colors that code ancillary directions are shown corresponding to the respective direction on the reference element. If primary colors such as red, green or blue are respectively specified for the primary directions, the intuitive comprehensibility of the color coding improves when combination colors resulting from the combination of the primary colors are used for the ancillary directions. For example, a lighter blue can be used for a vector that is somewhat inclined in comparison to the head-foot direction, the lighter blue slowly transitioning into green towards vectors that correspond more to an anterior-posterior direction.

Given a rotation of the reference element by means of an image processing tool, the representation of the quantities can be inventively rotated corresponding to the image output unit, and vice versa. The user thus has the possibility to use an image processing tool that he or she controls via a keyboard or mouse. For example, the reference element can be rotated as desired by the specification of a rotation angle and a rotation direction or directly by means of an arrow representation or a displayed grip hand. In this case, the image representation is adapted such that the orientation corresponds to that reproduced on the reference element. Reciprocally, the representation can be rotated on the image output unit, for example by the underlying parameters being adapted. In this case a corresponding rotation of the reference element ensues automatically, such that reference element reproduces the correct orientation with the associated color coding.

Using a single element, it is thus inventively possible to reproduce the orientation of an image representation together with an underlying color coding for shown vectorial quantities.

Furthermore, the invention concerns an apparatus for visualization of three-dimensional vectorial quantity with color coded direction information, having a data processing device for processing of data that are present and/or received data, and an image output unit suitable for color representation, the apparatus being designed for implementation of the method described above. The vectorial quantities are stored on the data processing device, or they are communicated to the data processing device (for example from a measurement device) via a data storage medium or a data connection. The image output unit of the apparatus, which can be a monitor or a flat screen or the like, is suited to represent the vectorial quantities with color coding. The reference element designed in color (and thus also indicating the color coding in addition to the orientation) is likewise shown. This typically occurs by means of a program stored in the data processing device for processing of data such as measurement or simulation data or, respectively, for image processing.

The apparatus includes an input device (in particular a keyboard and/or mouse) for operation of an image processing tool. For example, the reference element can be gripped and rotated by means of an image processing tool, and the control can ensue via a mouse or the like. The input device can additionally be used to control image processing via text inputs, by (for example) parameters that determine the orientation of the representation being changed. A selection of image regions or slice planes for a new representation can likewise ensue with the input device.

The invention also concerns a magnetic resonance system that has an apparatus for visualization of three-dimensional vectorial magnetic resonance quantities as described above. One possible quantity is the vector of the predominant direction of the diffusion that results in diffusion tensor imaging due to the different mobility of protons, or the anisotropy that results therefrom, this anisotropy defines a predominant direction. For example, the movement direction of the myocardium can be reproduced by vectorial quantities. Due to the use of the inventive apparatus, a more compact representation that prevents occlusions, but that is very intuitive results for the user who implements an examination with the magnetic resonance system.

### DESCRIPTION OF THE DRAWINGS

FIG. 1 shows a screen display in the framework of an inventive method for visualization of vectorial quantities.

FIG. 2 is a further image representation with a spherical reference element in accordance with the invention.

FIG. 2a shows a representation of a section of FIG. 2;

FIG. 3 shows a reference element designed according to the invention.

FIG. 4 illustrates an inventive magnetic resonance system with an apparatus for implementation of the inventive method.

### DESCRIPTION OF THE PREFERRED EMBODIMENTS

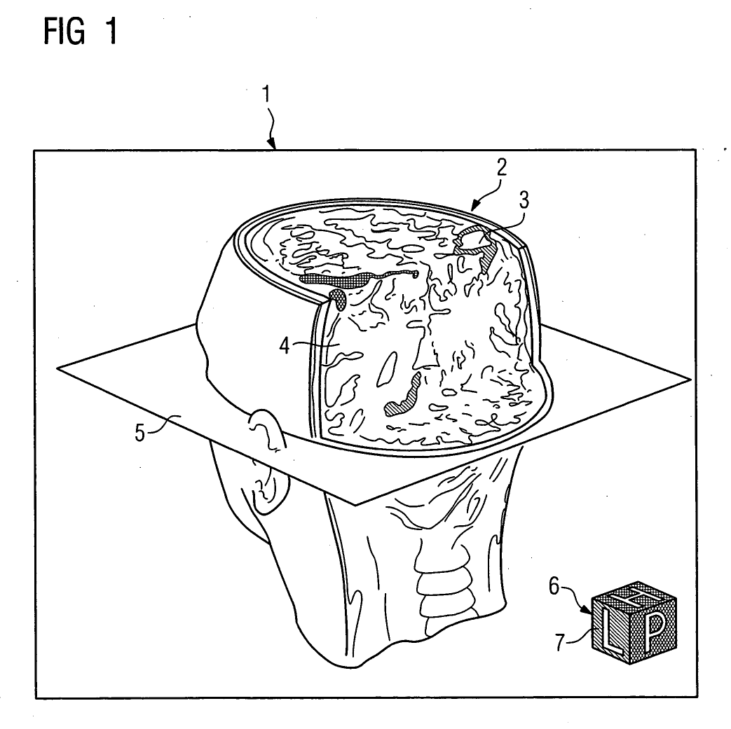

FIG. 1 shows a screen display 1 in the framework of an inventive method for visualization of three-dimensional vectorial quantities that are present at a data processing device or have been received thereby via a data line or by means of a storage medium. Alternatively or complementarily, an image printout (not shown here) can be considered. The vectorial quantities that were determined given a data acquisition in the medical field are reproduced in a three-dimensional representation 2 in the screen display 1. In addition to the representation 3 of the vectorial quantities that is color-coded dependent on the direction of the respective vectors, the three-dimensional representation 2 also includes a representation 4 of an associated examination region (here the head region of an examined patient) that serves for reference purposes. For this purpose, the head of the patient is shown with the corresponding skin or fatty tissue, the brain mass as well as skeleton. Furthermore, a plane 5 that intersects the head region of the three-dimensional representation 2 in the horizontal direction is reproduced for orientation.

In addition to the three-dimensional representation 2, the screen display 1 has a reference element 6 in a cube shape. The sides of the cube are provided with the text elements “L” for “left” as well as “P” for posterior and “H” for “head” that indicate the orientation of the anatomical representation 2. As indicated here by the different hatching, the sides 7 of the cube are patterned with different colors, whereby the color coding used for the representation 3 of the vectorial quantities is specified in an explanatory manner. Vectorial quantities whose direction corresponds to the posterior direction exhibit the same color (here to be recognized by the hatching) as the corresponding side 7 (identified with “P”) of the reference element 6. Vectors with a direction that corresponds to the left direction are accordingly reproduced in the representation 3 of the vectorial quantities with the same color as is indicated on the associated side 7 of the cube 6 that is identified with “L”. Quantities whose direction corresponds to none of these three primary directions standing orthogonal to one another are reproduced in the representation 3 by combination colors indicated via various hatchings.

With the inventive visualization method, it is thus possible to represent the orientation as well as the color coding that codes direction information of the vectorial quantities such that they can be comprehended quickly using a single reference element that is designed with corresponding color.

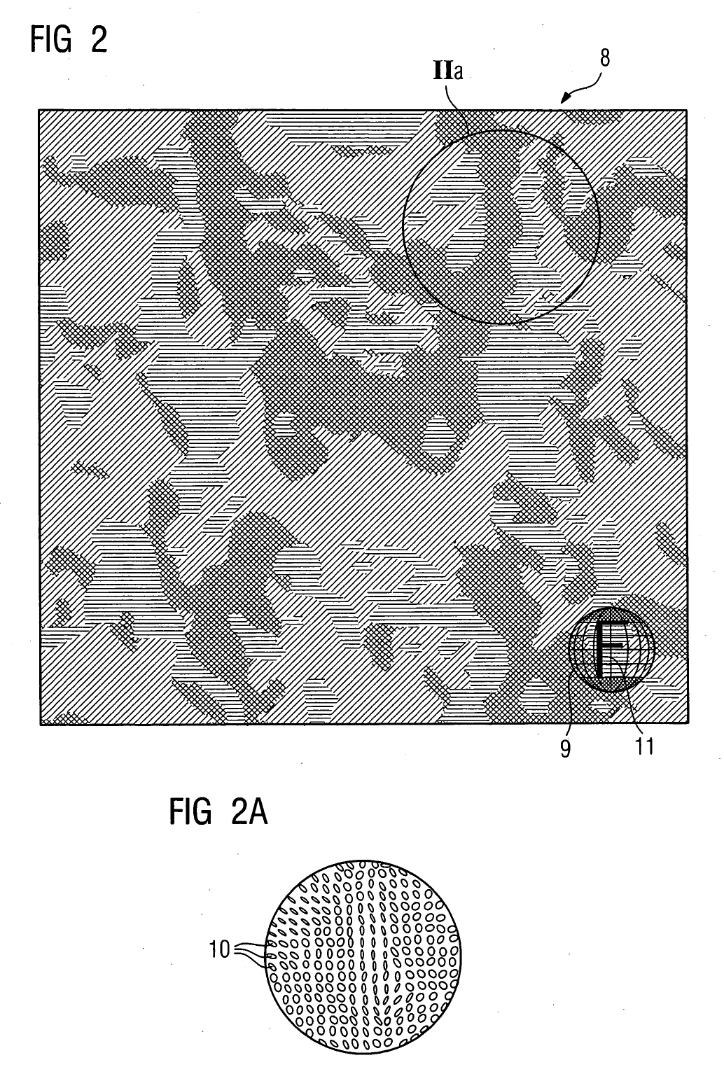

FIG. 2 shows a further screen display 8 with a spherical reference element 9. As is to be recognized in the section FIG. 2a, in this screen display 8 the vectorial quantities are shown two-dimensionally as ellipses 10, whereby the ratio of the semi-major axes of the ellipses 10 supplies a measure of the anisotropy of the quantity. The direction information of the vectorial quantities is reproduced by the color design of the ellipses 10, which here is indicated by different hatchings. For clarity, the ellipse structure is not shown in the representation 8; only the color regions corresponding to the hatchings are reproduced. This color coding is in turn explained by the color design of the reference element 9, which initially specifies the orientation of the representation 8 formed by the ellipses 10. For this purpose, the reference element 9 in the form of a sphere is provided with text elements 11, here “F” for “foot” and “A” for “anterior”, since here medical data were again acquired with which an anatomical orientation is to be appropriately associated. These text elements are patterned in color like the color coding for the respectively associated primary direction. Ancillary directions are coded via combination colors. Ellipses 10 whose coloring (here hatching in the representation) corresponds to a corresponding coloring (or hatching) on the reference element 9 exhibit the corresponding direction shown via the orientation of the reference element 9.

If, in FIGS. 1 or 2, the reference elements are rotated by the use of a suitable image processing tool, or by input of a specification determining the representation, a rotation of the representation of the vectorial quantities and, if applicable, of a shown examination region correspondingly ensues. Reciprocally, given a rotation of the image representation the reference element is rotated as well, such that the specifications that this supplies regarding orientation always correspond to the representation of the vectorial quantities.

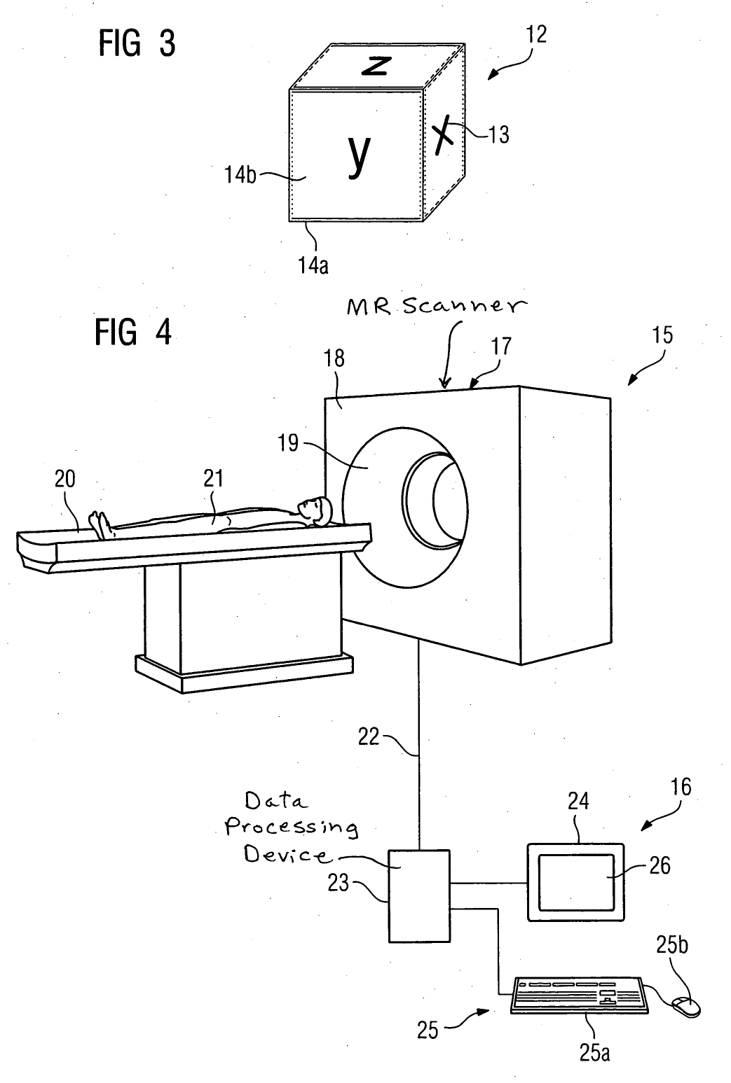

FIG. 3 shows a reference element 12 in the form of a cube, designed according to the invention. The orientation is shown here by the text elements 13 that refer to the x-, y- and z-directions, respectively. These primary directions form an orthogonal trihedron. The reference element 12 furthermore contains an explanatory specification regarding a color coding for vectorial quantities (not shown), in that the edges 14a of the reference element 12 are designed with different colors depending on their orientation in space, here shown via a corresponding dash or pattern and dot pattern. It is immediately discerned from the reference element 12 which color a vectorial quantity with a vertical predominant direction would have to have in an associated representation. The surfaces 14b are not patterned in color in the reference element 12 shown here, but rather exhibit a uniform coloration (for example gray) that is not to be confused with the color coding.

An inventive magnetic resonance system 15 with a device 16 for implementation of the inventive method is shown in FIG. 4. The magnetic resonance system 15 has a magnetic resonance scanner 17 that has a housing 18 as well as an opening 19 that is suitable for acceptance of a patient bed 20 on which a patient 21 is located.

With the magnetic resonance scanner 17, an image exposure in which vectorial quantities are measured with the diffusion directions is acquired in the framework of a diffusion tensor imaging. These vectorial quantities are transmitted to the apparatus 16 via the data connection 22, the apparatus 16 having a data processing device 23 with a storage and computation unit as well as a screen 24 and an input device 25 formed by a keyboard 25a as well as a mouse 25b. The vectorial quantities received from the magnetic resonance scanner 17 over the data connection 22 are processed by the data processing device 23 such that they are displayed color-coded on the screen 24 in a display 26 that also includes a reference element. The reference element (which is not shown for clarity) is fashioned such that it reproduces both the orientation of the three-dimensionally or, respectively, two-dimensionally projected representation of an acquired examination region and the color coding with whose help direction information of the vectorial quantities is coded. The orientation can be appropriately indicated by text components that moreover can simultaneously serve for specification of the color coding, in that they are designed colored. Furthermore, corresponding surfaces of the reference element can be designed colored.

A visualization of three-dimensional vectorial quantities with color-coded direction information is thus possible with the inventive magnetic resonance system 15 using the apparatus 16, such that only a single reference element is required for clarification of the significant properties of the visualization (namely the orientation or, respectively, the color coding). The information required to understand the data visualization is thus provided in a simple manner by the inventive magnetic resonance system 15.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method for visualization of three-dimensional vectorial quantities at a data processor, comprising the steps of:
at an image output associated with said data processor, automatically electronically causing said vectorial quantities to be represented in respectively different colors in a multi-dimensionally projected color-coded representation; and
together with said multi-dimensionally projected representation at said image output, automatically visually representing at least one reference element indicating an orientation of said representation and at least one explanatory specification for said color coding, said at least one reference element specifying said orientation of said representation in color as a three-dimensional element that is automatically generated by a program stored in said data processor, said color design of said reference element clarifying the color coding of said representation and thereby combining, in said reference element, said specification of said orientation and a three-dimensional explanation of said color coding in a single reference element.

2. A method as claimed in claim 1 comprising supplying said data processor with data obtained from diffusion tensor imaging of a diffusion process in an examination subject with a magnetic resonance imaging system and, in said multi-dimensionally projected color coded representation of said vectorial quantities, indicating a direction of said diffusion process as one of said vectorial quantities.

3. A method as claimed in claim 1 comprising representing said single reference element at said image output with a shape selected from the group consisting of cubes, spheres, cuboids and ellipsoids.

4. A method as claimed in claim 1 wherein the step of providing said reference element with a color design comprises representing different physical characteristics of said reference element in different colors, said physical characteristics being selected from the group consisting of edges of said reference element, complete surfaces of said reference element, and segmented surfaces of said reference element.

5. A method as claimed in claim 1 comprising embodying text components in said reference element at said image output that specify said orientation of said representation, and coloring said text components consistently with said color coding.

6. A method as claimed in claim 1 wherein said multi-dimensionally projected color coded representation comprises primary directions, and comprising color coding said primary directions respectively with primary colors.

7. A method as claimed in claim 6 comprising orienting said primary directions corresponding to an orthogonal trihedron.

8. A method as claimed in claim 6 comprising blending said primary colors in said multi-dimensionally projected color coded representation to represent ancillary directions between said primary directions in said reference element.

9. A method as claimed in claim 1 comprising rotating said reference element at said image output using an image processing tool of said data processor and correspondingly rotating said multi-dimensionally projected color-coded representation at said image output.

10. A method as claimed in claim 1 comprising rotating said multi-dimensionally projected color-coded representation at said image output using an image processing tool of said data processor, and correspondingly rotating said reference element at said image output.

11. An apparatus for visualization of three-dimensional vectorial quantities at a data processor, comprising:
a data processor having an image output at which said data processor automatically causes said vectorial quantities to be represented in respectively different colors in a multi-dimensionally projected color-coded representation; and
said data processor, together with said multi-dimensionally projected representation at said image output, automatically visually representing at least one reference element indicating an orientation of said representation and at least one explanatory specification for said color coding, said at least one reference element specifying said orientation of said representation in color as a three-dimensional element that is automatically generated by a program stored in said data processor, said color design of said reference element clarifying the color coding of said representation and thereby combining, in said reference element, said specification of said orientation and a three-dimensional explanation of said color coding in a single reference element.

12. An apparatus as claimed in claim 11 wherein said data processor employs an image processing tool to rotate said reference element at said image output, and correspondingly rotates said multi-dimensionally projected color-coded representation at said image output.

13. An apparatus as claimed in claim 11 wherein said data processor employs an image processing tool to rotate said multi-dimensionally projected color-coded representation at said image output, and correspondingly rotates said reference element at said image output.

14. A magnetic resonance system comprising:
a magnetic resonance scanner adapted to interact with an examination subject to obtain image data therefrom; and
a data processor supplied with said image data for visualization of three-dimensional vectorial quantities, embodied in said image data, at an image output associated with said data processor, said data processor automatically causing said vectorial quantities to be represented in respectively different colors in a multi-dimensionally projected color-coded representation, and, together with said multi-dimensionally projected representation at said image output, said data processor automatically visually representing at least one reference element indicating an orientation of said representation and at least one explanatory specification for said color coding, said at least one reference element specifying said orientation of said representation in color as a three-dimensional element that is automatically generated by a program stored in said data processor, said color design of said reference element clarifying the color coding of said representation and thereby combining, in said reference element, said specification of said orientation and a three-dimensional explanation of said color coding in a single reference element.
